(19) United States
(12) Patent Application Publication
(10) Pub. No.: US 2024/0017044 A1
(43) Pub. Date: Jan. 18, 2024

(54) BALLOON CATHETER SYSTEM

(71) Applicant: Togo Medikit Co.,Ltd., Tokyo (JP)

(72) Inventors: Yasuaki KAJIWARA, Tokyo (JP); Tomikazu FUKUMOTO, Tokyo (JP)

(21) Appl. No.: 18/256,782

(22) PCT Filed: Dec. 14, 2021
(86) PCT No.: PCT/JP2021/046143

§ 371 Date: Jun. 9, 2023

(30) Foreign Application Priority Data
Feb. 12, 2021 (JP) 2021-020513

(51) Int. Cl. A61M 25/09

(52) U.S. Cl. CPC A61M 25/09; A61M 2025/09125; A61M 25/10182

(57) ABSTRACT

The balloon catheter system has a balloon catheter having a balloon placed in the front end region, a one-way cock being detachably connected with the back end of the balloon catheter, and a syringe detachably attached to the one-way cock. The syringe inflates and deflates the balloon of the balloon catheter. The syringe has a cylindrical barrel being provided with one projection in the longitudinal direction of an inner periphery and a plunger is slidably placed in a longitudinal direction in the barrel. The plunger being provided with a circular concave connecting to the projection.

### CROSS-REFERENCE TO RELATED APPLICATIONS

This U.S. National Stage application claims the benefit and priority under 35 U.S.C. § 371 of PCT/JP2021/046143 filed on Dec. 14, 2021, which claims the benefit and priority of Japanese Patent application No. 2021-020513 filed on Feb. 12, 2021, the disclosures of which are incorporated herein by reference in their entireties.

### TECHNICAL FIELD

The present invention relates to a balloon catheter system. More specifically, the present invention relates to a balloon catheter system having a balloon catheter and a device for balloon inflation being attached to the back end of the balloon catheter through a cock, the device for balloon inflation inflating and deflating a balloon of the balloon catheter.

### BACKGROUND ART

Conventionally, the “inflation device” disclosed in Non-Patent Document 1 has been known as a device for balloon inflation used for this kind of a balloon catheter system. This inflation device is provided with a syringe outer cylinder (barrel), a plunger handle (piston), a latch (lock lever), a pressure gauge, and a connecting tube connected with a balloon catheter through a three-way cock. To pressurize and inflate the balloon, the latch is set to the central position, the plunger handle is locked, and the plunger handle is slowly rotated clockwise until it reaches a preferred pressure. On the other hand, to depressurize and deflate the balloon, the latch is turn to the left, the plunger handle is unlocked and pulled, and the latch is returned to the central position, and the plunger handle is locked. However, there is a problem where the inflation device disclosed in Non-Patent Document 1 is expensive. Moreover, this inflation device has a syringe outer cylinder (barrel) with a large inside diameter of approximately 28 mm so that a high pressure can be applied by rotating the plunger handle. This may cause an excessive pressure to be applied to the balloon without adequate confirmation of the pressure gauge. Moreover, the device with a capability of applying a high pressure, which typically has a limiter function that changes the maximum pressure to be applied for safety, may also cause an excessive pressure to be applied to the balloon by mistaking in setting this limiter.

As a device for balloon inflation, a “syringe assembly” with a relatively small barrel inside diameter as disclosed in Patent Document 1 is proposed. This syringe assembly is provided with a barrel, a plunger including a rib, and a fixing mechanism including a fixing surface being engageable with the rib. This syringe assembly is configured to be placed in a unfixed (unlocked) configuration in which the plunger is movable in a longitudinal direction in the barrel and a fixed (locked) configuration in which the plunger is immovable in a longitudinal direction in the barrel. In the fixed (locked) configuration, the rib is engaged with the fixing surface. The syringe assembly changes from the unfixed (unlocked) configuration to the fixed (locked) configuration, and vice versa by axially rotating the plunger in the barrel.

### DOCUMENT IN THE EXISTING ART

### Non-Patent Document

Non-Patent Document 1: KANEKA CORPORATION, “KANEKA inflation DEVICE”, General medical device, Pressurizer for angioplasty balloons, revised in June, 2017, 3rd edition, [online], retrieved on Dec. 4, 2020, Internet <URL;https://www.info.pmda.go.jp/downfiles/md/PDF/200095/200095_2 7B1X00034000001_A_01_03.pdf>

### Patent Document

Patent Document 1: JP 2016-512145 A

### SUMMARY

### The Technical Problem Solved by the Disclosure

The constitution of the syringe assembly disclosed in Patent Document 1 has a problem in which extra rotating operation of the plunger is required to lock and unlock the plunger. Moreover, this constitution has another problem in which the configuration of the fixing mechanism is complicated to involve a manufacturing cost.

An objective of the present invention is to provide a balloon catheter system at low cost, which causes no possible excessive pressure to be applied to the balloon and requires no rotating operation of the plunger to lock and unlock the plunger.

### Solution for Solving the Technical Problem

To achieve the objective, a balloon catheter system according to a first aspect of the present invention includes:
a balloon catheter having a balloon placed in a front end region;
a cock being detachably connected with a back end of the balloon catheter; and
a syringe being detachably attached to the cock, the syringe inflating and deflating the balloon of the balloon catheter, in which
the balloon catheter system is used to assist insertion and/or removal of an OTW (Over The Wire) catheter by inflating the balloon of the balloon catheter inserted in a guiding catheter and biasing and fixing a guide wire to an inner periphery of the guiding catheter,
the syringe has a cylindrical barrel being provided with a projection on the way of a longitudinal direction of an inner periphery and a plunger being slidably placed in a longitudinal direction in the barrel, the plunger being provided with a circular plate in a back end side forming a circular concave connecting to the projection,
the plunger is slid to a back end side of the barrel and moved to a first position in which the circular plate contacts the projection to allow a necessary amount of a balloon inflation medium to be introduced in the barrel to inflate the balloon and fix the guide wire,
the circular concave is placed to connect to the projection by sliding the plunger to a back end side of the barrel further than the first position,
the plunger is slid to a front end side of the barrel in a state in which the syringe containing the balloon inflation medium is connected with the balloon catheter to introduce the balloon inflation medium for inflating the balloon, and
the plunger is locked until the balloon inflation medium is removed in a state in which a negative pressure is applied by pulling the plunger to a second position at which the circular concave is connected to the projection to deflate the balloon after the guide wire is biased and fixed to an inner periphery of the guiding catheter, the second position being in a back end side further than the first position.

The balloon catheter system according the first aspect of the present invention has the following function effects. According to the first aspect of the present invention, the balloon catheter system applies a pressure by sliding the plunger in a longitudinal direction as the device for balloon inflation but does not apply high pressure by rotating the plunger handle unlike Non-Patent Document 1. This may not cause an excessive pressure to be applied to the balloon. As the result, it is possible to inflate the balloon safely. Moreover, the circular concave is connected to or disconnected from the projection only by sliding the plunger in the barrel so that the plunger can be locked or unlocked. As the result, it is possible to lock or unlock the plunger without rotating operation. Moreover, the lock mechanism of this plunger is simply composed of only a projection placed on the way in a longitudinal direction of the inner periphery of the barrel and a circular concave provided on the plunger, the circular concave being connected to the projection. This can suppress the manufacturing cost. Therefore, the configuration according to the first aspect of the present invention can provide a balloon catheter system at low cost, which causes no possible excessive pressure to be applied to the balloon and requires no rotating operation of the plunger to lock and unlock the plunger.

Moreover, according to the first aspect of the present invention, the (connecting) concave provided on the plunger is a circular concave so that the concave of the plunger can be connected to the projection of the barrel even if the plunger is rotated (in any phase). Moreover, according to the first aspect of the present invention, the circular concave is placed to connect to the projection by sliding the plunger to the back end side of the barrel. The plunger is pulled to the position of the projection to deflate the balloon so that the plunger can be left to locked until the balloon inflation medium is removed.

The balloon catheter system according to the first aspect of the present invention preferably has the following second or third aspect of the present invention.

According to the second aspect of the present invention, in the balloon catheter system according to the first aspect of the present invention, the circular concave is disconnected from the projection by moving the plunger backward or forward. The preferable configuration according to the second aspect of the present invention can easily unlock the plunger.

According to the third aspect of the present invention, in the balloon catheter system according to the first or the second aspect of the present invention, the balloon catheter is a trapping balloon catheter.

### Technical Effect

The present invention can provide a balloon catheter system at low cost, which causes no possible excessive pressure to be applied to the balloon and requires no rotating operation of the plunger to lock and unlock the plunger.

### BRIEF DESCRIPTION OF THE DRAWINGS

FIG. 1 is a side view illustrating the schematic configuration of the balloon catheter system according to one embodiment of the present invention. (The plunger is pushed in the front end of the barrel.)

FIG. 2 is a side view illustrating the schematic configuration of the syringe as one component of the balloon catheter system according to one embodiment of the present invention. (The one-way cock is attached, and the plunger is pushed in the front end of the barrel.).

FIG. 3 is a side view illustrating the schematic configuration of the syringe as one component of the balloon catheter system according to one embodiment of the present invention. (The plunger is pulled (to the position of the nominal capacity scale of the barrel) until the circular concave contacts the projection.)

FIG. 4 is a side view illustrating the schematic configuration of the syringe as one component of the balloon catheter system according to one embodiment of the present invention. (The circular concave is connected to the projection, and the plunger is locked.)

FIG. 5 is a side view illustrating the schematic configuration of the balloon catheter system according to one embodiment of the present invention. (The plunger is pulled (to the position of the nominal capacity scale of the barrel) until the circular concave contacts the projection.)

FIG. 6 is a side view illustrating the schematic configuration of the balloon catheter system according to one embodiment of the present invention. (The circular concave is connected to the projection, and the plunger is locked.)

FIG. 7 is a side view illustrating the state in which the catheter shaft of the balloon catheter as one component of the balloon catheter system according to one embodiment of the present invention is inserted in the lumen of the guiding catheter.

FIGS. 8A-8B are an enlarged cross-sectional side view to explain how to bias and fix a guide wire to the inner periphery of the guiding catheter by using the balloon catheter as one component of the balloon catheter system according to one embodiment of the present invention. (FIG. 8A The balloon is deflated. FIG. 8B The balloon is inflated.)

FIGS. 9A-9G are a process chart to explain how to remove a catheter by using the balloon catheter as one component of the balloon catheter system according to one embodiment of the present invention.

FIGS. 10H-10L are a process chart to explain how to insert a catheter (exchange catheters) by using the balloon catheter as one component of the balloon catheter system according to one embodiment of the present invention.

### DETAILED DESCRIPTION OF THE EMBODIMENTS

The present invention will be more specifically described below with reference to the preferable embodiments. However, these are illustrative only, and the present invention is not limited thereto.

### Configuration of Balloon Catheter

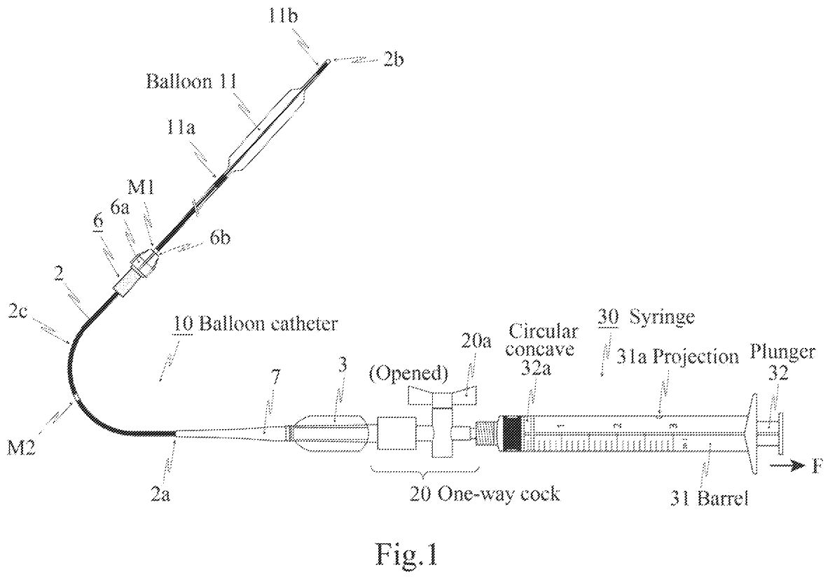

The configuration of the balloon catheter system according to one embodiment of the present invention is described below with reference to FIGS. 1 to 4. The size values (e.g., inside diameter and length) and the materials of each component described below are just one example.

The balloon catheter system shown in FIG. 1 has a balloon catheter 10 having a balloon 11 placed in the front end region, a (one-way) cock 20 being detachably connected with the back end of the balloon catheter 10, and a syringe 30 as a device for balloon inflation being detachably attached to the one-way cock 20, the syringe 30 inflating and deflating the balloon 11 of the balloon catheter 10.

As shown in FIGS. 1 to 4, the syringe 30 has a cylindrical barrel 31 being provided with one approximately hemispherical projection 31a on the way of a longitudinal direction of an inner periphery, the projection projecting radially inward, and a plunger 32 being slidably placed in a longitudinal direction in the barrel 31, the plunger 32 being provided with a circular concave 32a connecting to the projection 31a. In the embodiment, as a device for balloon inflation, the syringe 30 with a smaller barrel inside diameter is used rather than the syringe with a barrel inside diameter (approximate 28 mm) of Non-Patent Document 1. The height of the projection 31a is preferably from 1.4 to 1.6 mm, and the depth of the circular concave 32a is preferably from 3 to 5 mm. The height of the projection 31a and the depth of circular concave 32a that fall within their respective preferred ranges allow the circular concave 32a to be easily connected to and disconnected from the projection 31a. Therefore, the circular concave 32a can be connected to and disconnected from the projection 31a with no excessive but moderate power. In addition, the plunger can be appropriately locked without being carelessly unlocked.

The balloon catheter system according to the embodiment applies a pressure by sliding the plunger 32 in a longitudinal direction as the device for balloon inflation but does not apply high pressure by rotating the plunger handle unlike Non-Patent Document 1. This will not cause an excessive pressure to be applied to the balloon. As a result, it is possible to inflate the balloon 11 safely. Moreover, the circular concave 32a is connected to or disconnected from the projection 31a only by sliding the plunger 32 in the barrel 31 so that the plunger 32 can be locked or unlocked. As the result, it is possible to lock or unlock the plunger 32 without rotating operation. Moreover, the lock mechanism of this plunger 32 is simply composed of only a projection 31a placed in the longitudinal direction of the inner periphery of the barrel and a circular concave 32a provided on the plunger 32, the circular concave being connected to the projection 31a. This can suppress the manufacturing cost. Therefore, the configuration of the balloon catheter system according to the embodiment may not cause an excessive pressure to be applied to the balloon 11 and can provide the balloon catheter system at low cost, which requires no rotating operation of the plunger 32 to lock and unlock the plunger 32.

Moreover, according to this configuration, the (connecting) concave provided on the plunger 32 is a circular concave 32a so that the concavity of the plunger 32 can be connected to the projection 31a of the barrel 31 even if the plunger 32 is rotated (in any phase).

More details are described below. The barrel 31 shown in FIGS. 1 to 4 has a cylindrical form with a length in a longitudinal direction of approximate 60 mm and an inside diameter of approximate 9.70 mm. At the front end of the barrel 31, a clysis inlet 31b and a cylindrical joint 31c formed to surround the outer periphery of the clysis inlet 31b are placed. The inner periphery of the joint 31c has a female thread. Moreover, a flange with a male thread (not shown in the attached drawings) is formed at one end of the one-way cock 20. Accordingly, the barrel 31 can be detachably threaded with one end of the one-way cock 20. A first flange 31d is placed at the back end of the barrel 31.

Two small circular plates 32b, 32c are placed at the front end of the plunger 32 in an axial direction at a predetermined interval to form a circular concave 32a. On the small circular plate 32b of the front end of the plunger 32, a gasket 32d slidably contacting the inner periphery of the barrel 31 with liquid tightness is fixed. A second flange 32e is placed at the back end of the plunger 32.

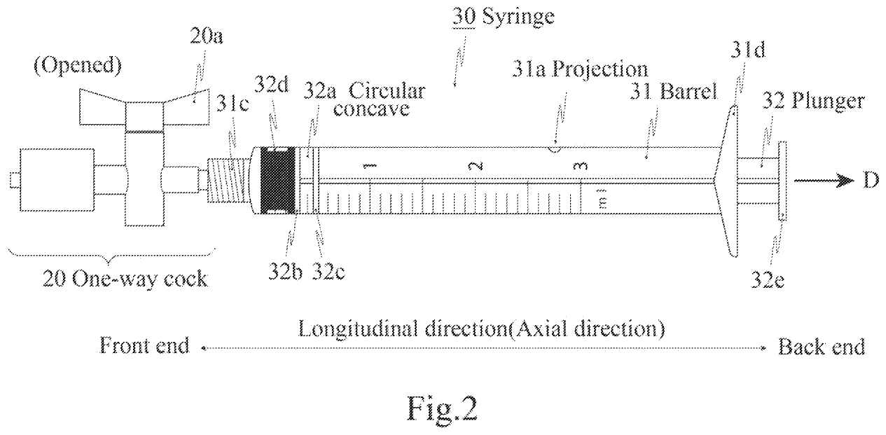

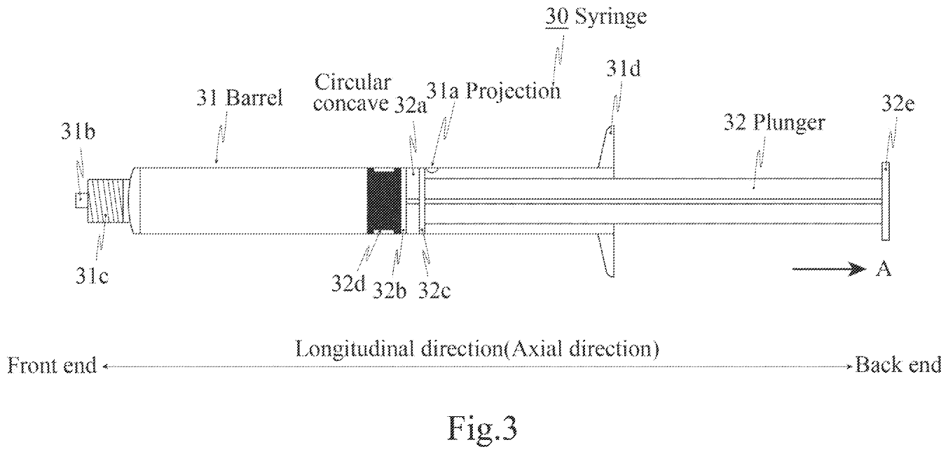

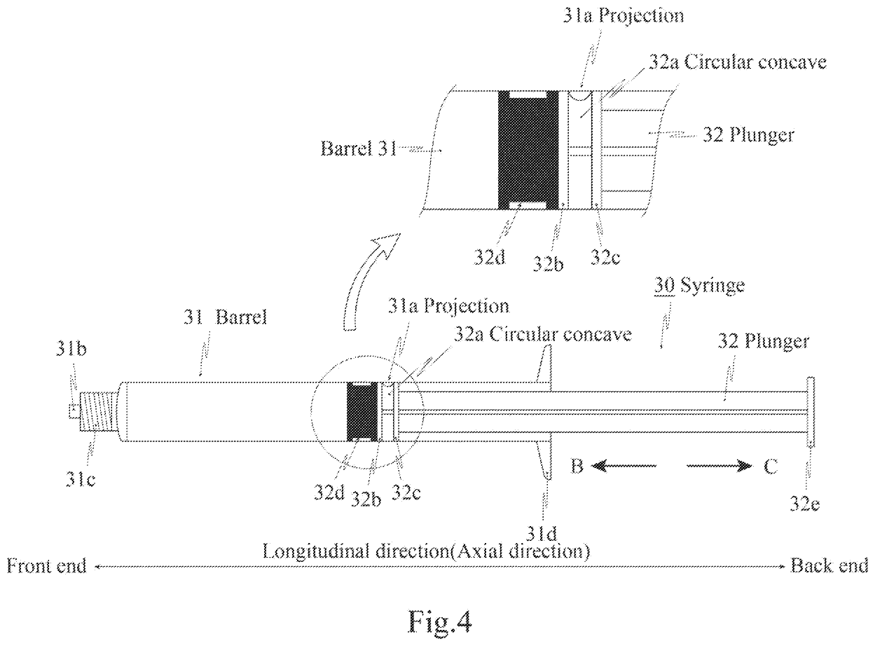

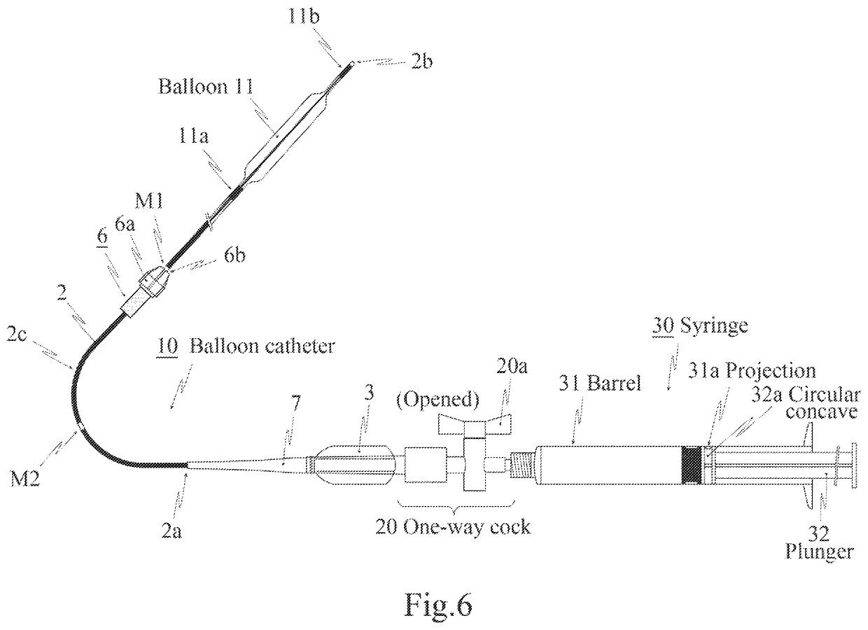

As shown in FIGS. 2 to 4, the projection 31a of the inner periphery of the barrel 31 and the circular concave 32a of the plunger 32 are placed to be connected with each other by sliding the plunger 32 to the back end side of the barrel 31 (refer to the arrow A of FIG. 3). According to this configuration, the plunger 32 is pulled to the position of the projection 31a to deflate the balloon 11 so that the plunger 32 can be left to the locked position until the balloon inflation medium is removed (as shown in FIG. 6). Moreover, the projection 31a on the inner periphery of the barrel 31 is placed so that the front end of the gasket 32d approximately comes to the position indicated by a scale of the nominal content (2 ml) of the barrel 31 when the projection 31a contacts the small circular plate 32c in the back end side that forms the circular concave 32a of the plunger 32 (as shown in FIG. 3). The projection 31a is disconnected from the circular concave 32a by moving the plunger 32 backward or forward (refer to the arrows B, C of FIG. 4). According to the configuration, the plunger 32 can be easily unlocked.

The barrel 31 is preferably formed from a transparent or a translucent resin material so that the inside can be at least viewed. The example materials of the barrel 31 include polyvinyl chloride, polyethylene, polypropylene, polystyrene, polymethylpentene, polycarbonate, polyamide, acrylic resins, polyester such as polyethylene terephthalate, and cyclic olefine resins. For example, the gasket 32d is formed from an elastic body such as a vulcanized rubber or a thermoplastic elastomer. The parts other than the gasket 32d of the plunger 32 is preferably formed from a rigid or semirigid resin such as a high-density polyethylene, polypropylene, polystyrene, a butadiene-styrene copolymer, an acrylonitrile-butadiene-styrene copolymer, polymethylpentene, polycarbonate, an acrylic resin, polyethylene terephthalate, or cyclic polyolefin.

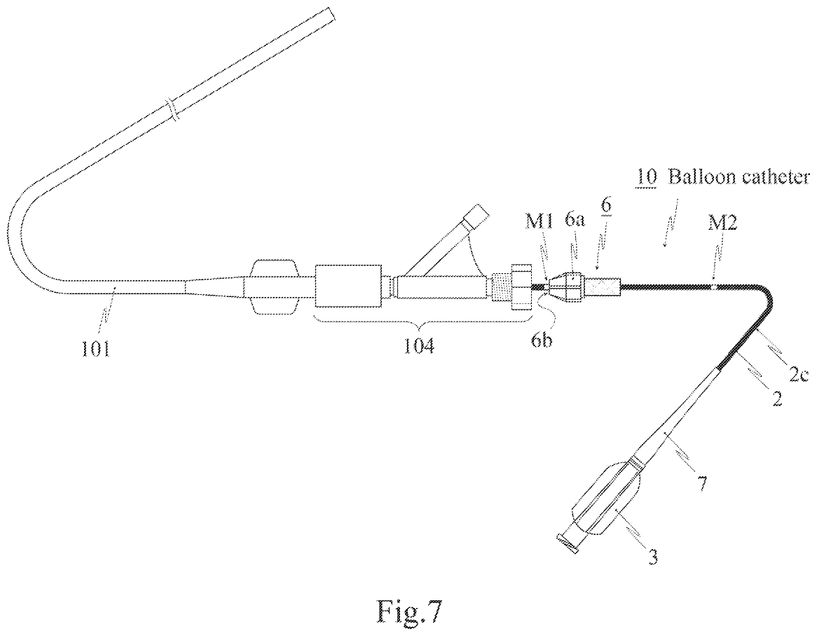

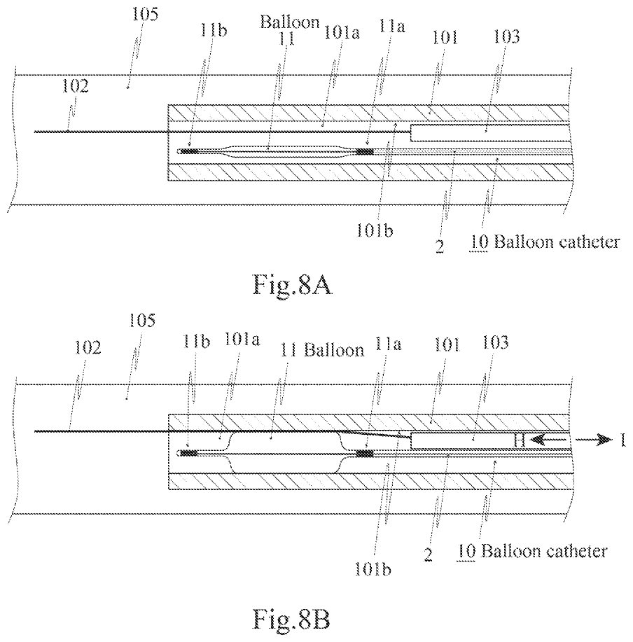

The balloon catheter 10 shown in FIG. 1 is a trapping balloon catheter 1 biasing and fixing a guide wire 102 to the inner periphery 101b of the guiding catheter 101 while the balloon catheter 10 is inserted in a lumen 101a of a guiding catheter 101 is used to assist insertion and/or removal of various catheters 103 such as a penetration catheter, a micro catheter, a balloon catheter and typical balloon catheters for treatment or diagnosis (refer to the arrows H, I of FIGS. 7, 8A, and 8B). The “balloon catheter 10” is sometimes hereinafter referred to as the “trapping balloon catheter 10”. The reference numeral 105 in FIGS. 8A to 10L shows a blood vessel.

The trapping balloon catheter 10 has a catheter shaft 2, the balloon 11 placed in the top end region of the catheter shaft 2, and a stopper 6 placed on the outer periphery 2c of the catheter shaft 2. A lumen for balloon inflation (not shown in the attached drawings) is formed on the catheter shaft 2 along the axial direction. The catheter shaft 2 is connected with the hub 3 through the strain relief (anti-kink protector) 7. The balloon 11 is inflatable by introducing a balloon inflating medium from the base end opening of the hub 3. The balloon 11 biases and fixes the guide wire 102 to the inner periphery 101b of the guiding catheter 101 by inflating in the lumen 101a of the guiding catheter 101 (refer to FIG. 8B) X-ray impermeable markers 11a and 11b are provided in the outside of both ends (the proximal side and the distal side) of the balloon 11 to allow the user to confirm the position of the balloon 11 under X-ray illumination.

The stopper 6 is simply provided with a lock mechanism, the lock mechanism being locked to fix at a predetermined position in a longitudinal direction of the catheter shaft 2, the lock mechanism 6d being unlocked to slidably move on the outer periphery 2c of the catheter shaft 2 in a longitudinal direction; a gripper 6a being gripped in back-and-forth moving operation and rotating operation of the trapping balloon catheter 10; and an abutting part 6b abutting the back end of a Y-shaped connector (connection device) 104 (refer to FIG. 7) placed at the base end of the guiding catheter 101. The insertion length of the trapping balloon catheter 10 into the lumen 101a of the guiding catheter 101 can be adjusted by changing the locked position. A position with a length of 90 cm from the front end 2b of the catheter shaft 2 is marked with the marker M1, and a position with a length of 100 cm from the front end 2b of the catheter shaft 2 is marked with the marker M2. The top end of the stopper 6 is matched and fixed (locked) at either the marker M1 or M2 depending on the effective length of the guiding catheter 101 to be used (refer to FIG. 7).

The catheter shaft 2 is formed from a flexible material. Examples of the material of the catheter shaft 2 include stainless steel and polyamide. The outside diameter of the catheter shaft 2 is 1.7 Fr (0.55 mm), and the total length (from the back end 2a to the front end 2b (effective length)) is 1170 mm. The total length of the trapping balloon catheter 10 (from the back end of the hub 3 to the front end 2b of the catheter shaft 2) is 1264 mm. Specifically, the catheter shaft 2 of the embodiment, the part except the top end region is formed from stainless steel, and the top end region is formed from polyamide. In this case, PTFE coating (black color) is applied to the stainless steel (silver color) except the vicinity of the top end region and the markers M1 and M2 to show the top end region part and the markers M1 and M2 part more prominently than the other parts.

The balloon 11 is also formed from a flexible material. Examples of the material of the balloon 11 include polyamide. The recommended inflation pressure of the balloon 11 is 8 atm (0.8 MPa), and the maximum inflation pressure is 14 atm (1.4 MPa). The outside diameter of the balloon at the recommended inflation pressure is 2.75 mm, and the effective length of the balloon is 20 mm. For example, a diluted contrast agent (the mixture of a contrast agent and saline) is used as a balloon inflation medium.

### Use of Balloon Catheter System

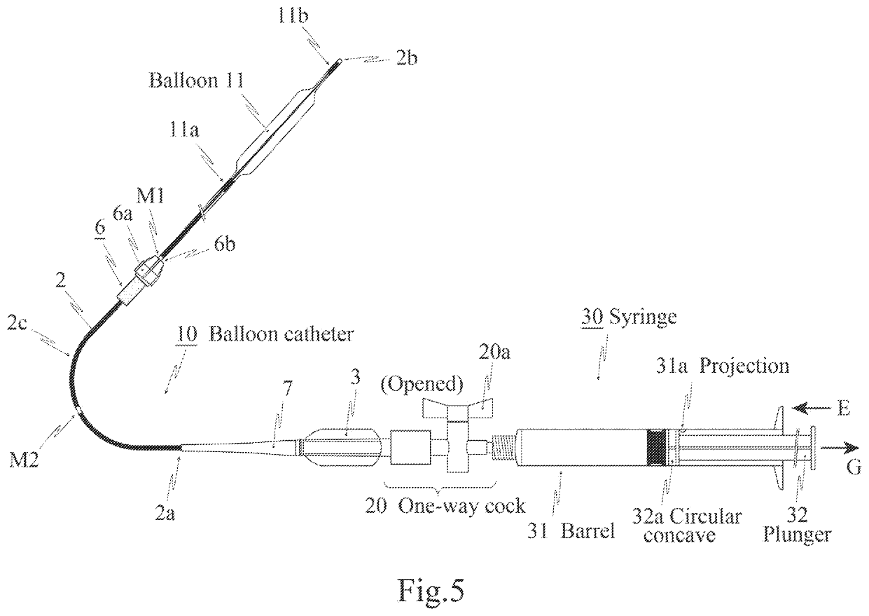

The use of the balloon catheter system according to one embodiment of the present invention is described below with reference to FIGS. 5 to 10L. The balloon catheter system of the embodiment is used to assist exchange of catheters in percutaneous transluminal coronary angioplasty (PTCA) which is a less invasive procedure using a catheter. In PTCA, a catheter for diagnosis and a catheter for treatment are exchanged for each other in a guiding catheter while a blood vessel, etc., is treated.

### Preparation Before Operation

Air is removed from the balloon 11 and the lumen for balloon inflation of the trapping balloon catheter 10 by following the procedure described below.
(a) As shown in FIG. 5, a syringe 30, in which a diluted contrast agent as a balloon inflation medium is injected is attached to the one-way cock 20 connected with the hub 3, and the front end 2b of the trapping balloon catheter 10 is aimed downward.
(b) After applying negative pressure enough by the syringe 30, the negative pressure is slowly released, and the balloon 11 and the lumen for balloon inflation of the trapping balloon catheter 10 are filled with the diluted contrast agent to remove the air.
(c) The above-mentioned step (b) is repeated to completely remove air from the balloon 11 and the lumen for balloon inflation of the trapping balloon catheter 10.
(d) The syringe for balloon inflation 30 is detached from the one-way cock 20 connected with the hub 3 to remove air from the syringe 30.
(e) The syringe 30 is attached to the one-way cock 20 connected with the hub 3 again, and negative pressure is applied. After it is confirmed that air does not return to the syringe 30, the negative pressure is slowly released.

### Insertion of Trapping Balloon Catheter, Exchange of Catheters, and Removal of Trapping Balloon Catheter

As shown in FIG. 1, the front end of the stopper 6 is matched and fixed (locked) at either the marker M1 or M2 depending on the effective length (90 cm or 100 cm) of the guiding catheter 101 to be used (refer to FIG. 7). As shown in FIG. 2, a one-way cock 20 is attached to the syringe 30 and inserted in the diluted contrast agent with which a sterilized container is filled. The plunger 32 is pulled (refer to the arrow D of FIG. 2) to bring the circular plate 32c in the back end side that forms the circular concave 32a into contact with the projection 31a as shown in FIG. 3. As the result, the diluted contrast agent with the nominal content (2 ml) is introduced in the syringe 30. To prevent the introducing of diluted contrast agent from being substituted with air, the cock 20a of the one-way cock 20 is closed.

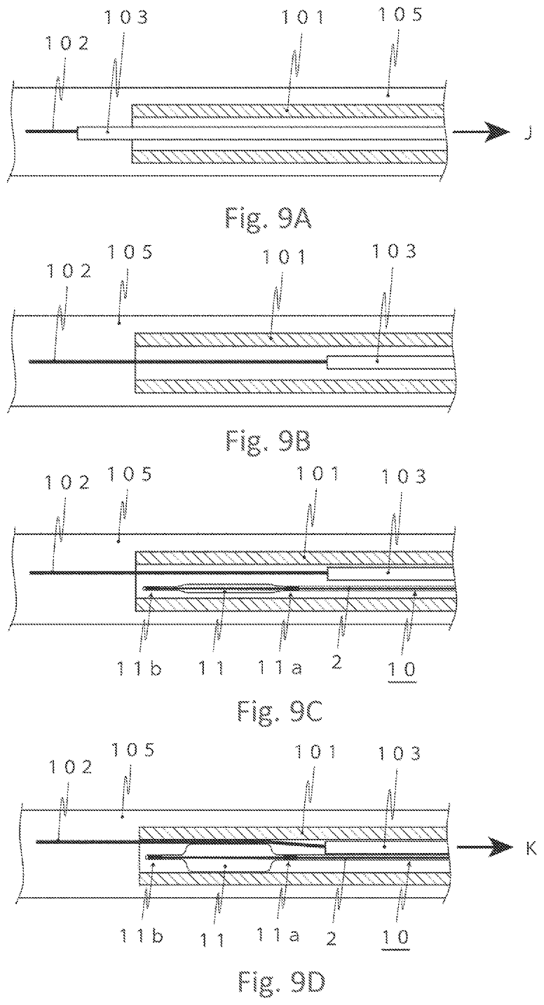

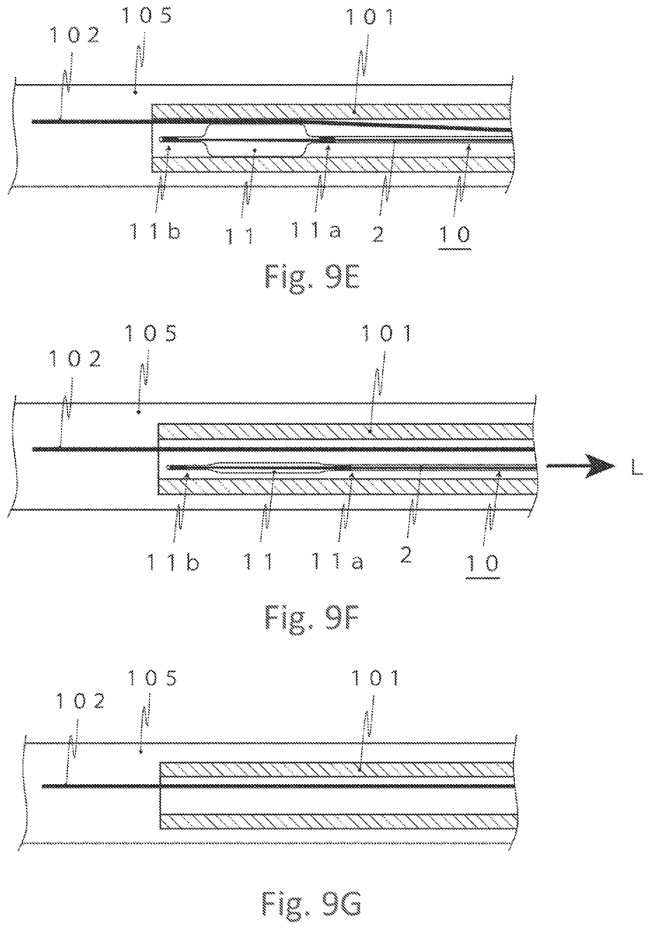

As shown in FIGS. 9A and 9B, while the guide wire 102 is fixed, the hub of a catheter 103 (e.g., OTW (Over The Wire) catheter) is drawn back enough into the guiding catheter 101 until it comes to the vicinity of the back end of the guide wire 102 (refer to the arrow J of FIG. 9A).

As shown in FIGS. 7, 8A, and 9C, while the OTW catheter 103 is fixed, the trapping balloon catheter 10 is inserted into the guiding catheter 101 under X-ray illumination, and the abutting part 6b of the stopper 6 is brought to abut the back end of a Y-shaped connector (connection device) 104 placed at the base end of the guiding catheter 101. At this time, it is confirmed that the X-ray impermeable marker 11a in the proximal side of the balloon 11 of the trapping balloon catheter 10 is distally located from the top end of the OTW catheter 103.

As shown in FIG. 5, the one-way cock 20 attached to the syringe 30 in which the diluted contrast agent is introduced is connected with the base end opening of the hub 3 of the trapping balloon catheter 10. As shown in FIGS. 5, 8B, and 9D, the cock 20a of the one-way cock 20 is opened, and the diluted contrast agent is injected with the syringe 30 (refer to the arrow E of FIG. 5) to inflate the balloon 11 of the trapping balloon catheter 10. As the result, the guide wire 102 is biased and fixed to the inner periphery 101b of the guiding catheter 101. At this time, the hand part of the guide wire 102 is lightly pulled to confirm that the guide wire 102 is fixed. If the guide wire 102 is not fixed enough, pressure is applied not to exceed the maximum inflation pressure of 14 atm (1.4 MPa) of the balloon 11 until the guide wire 102 is fixed enough. Once the guide wire 102 is fixed enough, the introduction of the diluted contrast agent is stopped, and the cock 20a of the one-way cock 20 is closed. In this embodiment, as a device for balloon inflation, the syringe 30 with a smaller barrel inside diameter (approximate 9.70 mm) is used rather than the syringe with a barrel inside diameter (approximate 28 mm) of Non-Patent Document 1. This will not cause an excessive pressure to be applied to the balloon 11. As a result, it is possible to inflate the balloon 11 safely.

As shown in FIGS. 9D and 9E, while it is observed under X-ray illumination that the position of the guide wire 102 is maintained, the OTW catheter 103 is slowly removed (refer to the arrow K of FIG. 9D).

As shown in FIGS. 1, 3, 5, 6, and 9F, the cock 20a of the one-way cock 20 is opened, and negative pressure is applied to the syringe 30 (refer to the arrow F of FIG. 1 and the arrow A of FIG. 3) to deflate the balloon 11 of the trapping balloon catheter 10. At this time, it is confirmed under X-ray illumination that the balloon 11 is deflated completely. Accordingly, to deflate the balloon 11, the plunger 32 is pulled to the position of the projection 31a (refer to the arrow F of FIG. 1 and the arrow A of the FIG. 3) and further pulled (refer to the arrow G of FIG. 5) to connect the circular concave 32a to the projection 31a so that the plunger 32 can be left to locked until the diluted contrast agent is removed (as shown in FIG. 6).

As shown in FIGS. 9F and 9G, the position of the guide wire 102 is maintained, and the trapping balloon catheter 10 is carefully, slowly removed from the guiding catheter 101 without excessive power applied while the balloon 11 of the trapping balloon catheter 10 is completely deflated (refer to the arrow L of FIG. 9F).

After it is confirmed that there is flashback from the Y-shaped connector (connection device) 104 shown in FIG. 7 to remove air from the guiding catheter 101, the following step is performed.

If insertion (exchange) of another OTW catheter or a monorail catheter is necessary after removal of the OTW catheter 103, the balloon 11 of the trapping balloon catheter 10 is inflated as shown in FIG. 9E to keep basing and fixing the guide wire 102 to the inner periphery 101b of the guiding catheter 101 (refer to FIGS. 8A-8B).

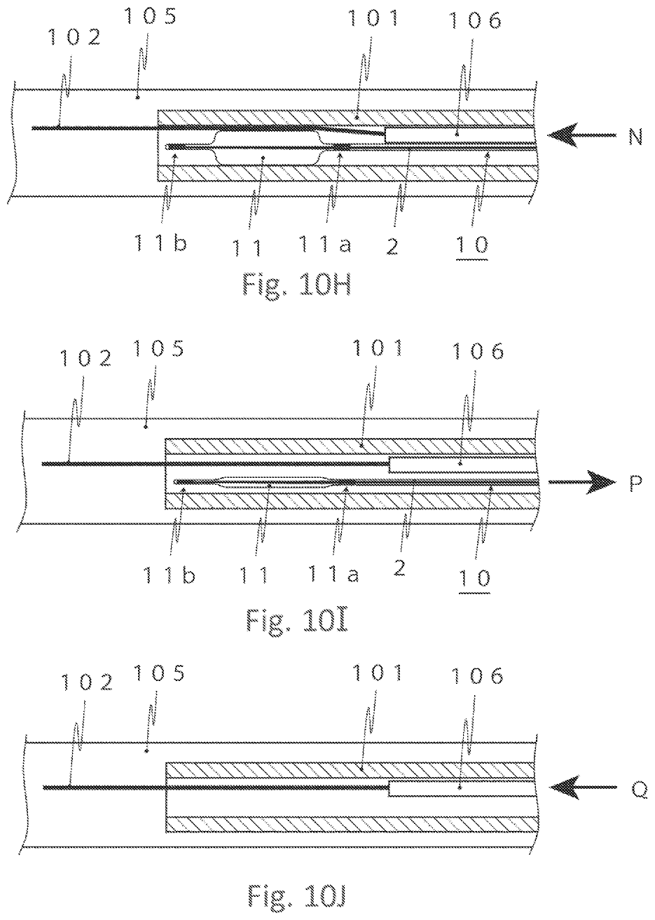

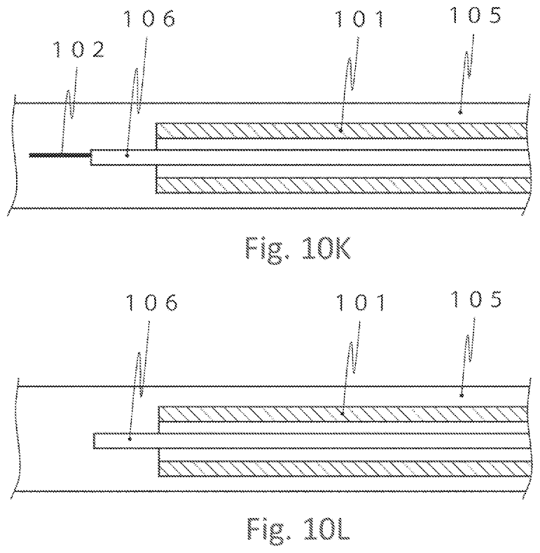

As shown in FIG. 10H, another OTW catheter 106 is slowly inserted into the vicinity of the X-ray impermeable marker 11a in the proximal side of the balloon 11 of the trapping balloon catheter 10 along the guide wire 102 (refer to the arrow N of FIG. 10H).

As shown in FIGS. 1, 3, 5, 6, and 10I, the cock 20a of the one-way cock 20 is opened, and negative pressure is applied to the syringe 30 (refer to the arrow F of FIG. 1 and the arrow A of FIG. 3) to deflate the balloon 11 of the trapping balloon catheter 10. At this time, it is confirmed under X-ray illumination that the balloon 11 is deflated completely.

As shown in FIGS. 10I and 10J, the position of the guide wire 102 is maintained, and the trapping balloon catheter 10 is carefully, slowly removed from the guiding catheter 101 without excessive power applied while the balloon 11 of the trapping balloon catheter 10 is completely deflated (refer to the arrow P of FIG. 10I).

As shown in FIGS. 10J and 10K, while the guide wire 102 is fixed, the OTW catheter 106 is pushed (refer to the arrow Q of FIG. 10J) to project the OTW catheter 106 from the top end of the guiding catheter 101.

As shown in FIGS. 10K and 10L, while the OTW catheter 106 is fixed, the guide wire 102 is slowly removed. The exchange of catheters is now completed.

The embodiment has been explained, giving an example where the syringe 30 has a barrel 31 with an inside diameter of approximate 9.70 mm. However, the present invention is not limited to such a configuration. The inside diameter of the barrel only has to fall within the range from 9.0 to 11.0 mm. The excessive pressure needed to destroy the balloon 11 of the balloon catheter 10 is about 20 atm (2.0 MPa). However, it is generally impossible for humans to apply the pressure if the inside diameter of the barrel falls within the range. This will not cause an excessive pressure to be applied to the balloon 11. As a result, it is possible to inflate the balloon 11 safely.

The embodiment has also been explained, giving an example where one approximately hemispherical projection 31a on the way in a longitudinal direction of the inner periphery of the barrel 31. However, the present invention is not limited to such a configuration. Two or more projections (e.g., two or three projections) may be placed along the circumferential direction of the inner periphery of the barrel. Moreover, the projection only has to has a form to connect to the circular concave and may be a circular projection, for example.

The embodiment has been explained, giving an example where the balloon catheter 10 is a trapping balloon catheter used to assist insertion and/or removal of various catheters. However, the present invention is not limited to such a configuration. For example, the balloon catheter 10 may be a balloon catheter for treatment or diagnosis.

### DESCRIPTION OF REFERENCE NUMERALS

2: catheter shaft, 2a: back end, 2b: front end, 2c: outer periphery, 3: hub, 6: stopper, 6a: gripper, 6b: abutting part, 7: strain relief (anti-kink protector), 10: balloon catheter (trapping balloon catheter), 11: balloon, 11a, 11b: X-ray impermeable marker, 20: (one-way) cock, 30: syringe, 31: barrel, 31a: projection, 31b: clysis inlet, 31c: joint, 31d: first flange, 32: plunger, 32a: circular concave, 32b, 32c: small circular plate, 32d: gasket, 32e: second flange, 101: guiding catheter, 101a: lumen, 101b: inner periphery, 102: guide wire, 103, 106: (OTW) catheter, 104: Y-shaped connector (connection device), 105: blood vessel

1. A balloon catheter system comprising:
a balloon catheter having a balloon placed in a front end region;
a cock being detachably connected with a back end of the balloon catheter; and
a syringe being detachably attached to the cock, the syringe inflating and deflating the balloon of the balloon catheter, the syringe having a cylindrical barrel with an inner periphery, a projection provided on the inner periphery, and a plunger being slidably placed in a longitudinal direction of the barrel, the plunger being provided with a circular plate in a back end side and forming a circular concavity configured to connect to the projection;
wherein the balloon catheter system is used to assist insertion and/or removal of an OTW (Over The Wire) catheter by inflating the balloon of the balloon catheter inserted in a guiding catheter and biasing and fixing a guide wire to an inner periphery of the guiding catheter,
wherein the plunger is slid to a back end side of the cylindrical barrel and moved to a first position in which the circular plate contacts the projection to allow a necessary amount of a balloon inflation medium to be introduced in the cylindrical barrel to inflate the balloon and fix the guide wire,
the circular concavity is positioned to connect to the projection by sliding the plunger toward a back end side of the cylindrical barrel further than a first position,
the plunger is slid to a front end side of the barrel in a state in which the syringe containing the balloon inflation medium is connected with the balloon catheter to introduce the balloon inflation medium for inflating the balloon, and
the plunger is locked until the balloon inflation medium is removed in a state in which a negative pressure is applied by pulling the plunger to a second position at which the circular concave is connected to the projection to deflate the balloon after the guide wire is biased and fixed to an inner periphery of the guiding catheter, the second position being in a back end side further than the first position.

2. The balloon catheter system according to claim 1, wherein the circular concavity is disconnected from the projection by moving the plunger backward or forward.

3. The balloon catheter system according to claim 1, wherein the balloon catheter is a trapping balloon catheter.
